(19) United States
(12) Patent Application Publication
(10) Pub. No.: US 2007/0190143 A1
(43) Pub. Date: Aug. 16, 2007

(54) Disintegrating tablets comprising licarb azepine

(76) Inventors: Oskar Kalb, Lorrach (DE); Marie-Christine Wolf, St. Louis (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/598,553

(22) PCT Filed: Mar. 21, 2005
(86) PCT No.: PCT/EP05/02987

§ 371 Date: Sep. 5, 2006

(30) Foreign Application Priority Data
Mar. 22, 2004 (GB) 0406381.4
Mar. 25, 2004 (GB) 0406737.7

(51) Int. Cl. A61K 9/26

(52) U.S. Cl. 424/470; 424/468

(57) ABSTRACT

The invention relates to pharmaceutical compositions comprising 10,11-dihydro-10-hydroxy-5H-dibenz[b,f]azepine-5-carboxamide (also referred to as “licarbazepine”) as drug substance.

The present invention relates to pharmaceutical compositions comprising 10,11-dihydro-10-hydroxy-5H-dibenz[b,f]azepine-5-carboxamide (also referred to herein as “licarbazepine”) as drug substance.

The term licarbazepine as used herein refers to the racemic mixture of (S)-10,11-dihydro-10-hydroxy-5H-dibenz[b,f]azepine-5-carboxamide and (R)-10,11-dihydro-10-hydroxy-5H-dibenz[b,f]azepine-5-carboxamide.

In the present invention licarbazepine, mixtures of (S)-10,11-dihydro-10-hydroxy-5H-dibenz[b,f]azepine-5-carboxamide and (R)-10,11-dihydro-10-hydroxy-5H-dibenz[b,f]azepine-5-carboxamide comprising one of the two enantiomers in excess, or one of the essentially pure or pure enantiomers of licarbazepine can be employed as drug substance and are all together hereinafter referred to as the “compounds of the invention”.

Licarbazepine (also known as MHD) is well known from the literature [see, for example, Schuetz H. et al., Xenobiotica (GB), 16(8), 769-778 (1986)] and can be prepared synthetically, for example starting from oxcarbazepine, according to conventional methods, e. g. as described in U.S. Pat. No. 3,637,661.

The pure enantiomers of licarbazepine can be obtained starting from the racemate by procedures known as such. For instance, the racemate may be separated into its enantiomers through the formation of diastereomers, e. g. as disclosed in WO-02/092572, or, alternatively, by salt formation with an enantiomer-pure chiral acid, or by means of chromatography, for example by HPLC, using chromatographic substrates with chiral ligands. In one embodiment of the invention, the pure enantiomers of licarbazepine are prepared by an enantioselective process described in the Examples.

Licarbazepine is indicated to be suitable for the treatment of psychosomatic disturbances, epilepsy, trigeminal neuralgia and cerebral spasticity. It was demonstrated that the racemate of licarbazepine and both of its pure enantiomers are of equal efficacy against epilepsy. The mechanisms by which the compounds of the invention exert their anticonvulsant effects are not completely understood, but their activity may be partly due to effects on ion flow across neuronal membranes. However, pharmacokinetics, absorption sites and mechanisms of action of the compounds of the invention are not understood in detail. Licarbazepine is slightly soluble in water (3.2 mg/ml at 25° C.). In view of this physical property, a parenteral formulation of licarbazepine can be prepared as described, e. g., in EP-1 033 988. Despite the merits of the known parenteral dosage form, there remains a need to establish an advantageous oral dosage form of the compounds of the invention. One of the problems that may occur using an oral dosage form is the fluctuation of blood levels of the compounds of the invention on repeated administration, which may be associated with side effects.

After exhaustive testing, advantageous pharmaceutical oral controlled release compositions, which are capable of being administered once a day and which are particularly well tolerated and have a good bioavailability in a wide variety of patient populations, have now surprisingly been found.

Hence, in one aspect the present invention relates to pharmaceutical oral controlled release compositions adapted to be administered once a day comprising at least one of the compounds of the invention (hereinafter referred to as “oral dosage forms of the invention”), in particular showing a low fluctuation index for a better tolerability and a continuous symptom control with an adequate Cₘᵢₙ (Minimum Plasma Concentration) value and furthermore having the advantage of a high AUC (Area Under the Curve) and a low Cₘₐₓ (Maximum Plasma Concentration) value.

The oral dosage forms of the invention may represent a considerable advantage over other oral dosage forms in that they are more convenient and/or safer for patients to use and increase the patients' compliance to therapy. The patients have to take the oral dosage forms of the invention only once a day.

The term “once a day” as used herein means once every 20 to 28 hours, in particular once every 24 hours.

Preferred oral dosage forms of the invention have the form of disintegrating tablets with modified release granules comprising the compounds of the invention, especially licarbazepine. In such oral dosage forms, the compounds of the invention, especially licarbazepine, can be present in the modified release granules in an amount of from 60 to 90%, preferably from 75 to 85%, e. g. in an amount of about 80%, by weight of the modified release granules or in an amount of from 50 to 80%, preferably from 60 to 70%, e. g. in an amount of about 65%, by weight of the total composition.

The compounds of the invention, especially licarbazepine, are preferably employed in coarse form, i. e. having a median particle size (x₅₀) of from about 150 to about 300 μm, preferably from about 200 to about 250 μm, more preferably from about 210 to about 230 μm, e. g. of about 220 μm.

In one embodiment of the invention, the oral dosage form comprises in the modified release granules at least one retarding agent selected from the group of compounds, consisting of polymethacrylates, ethylcellulose, hydroxyethylcellulose, hydroxypropylcellulose and methylcellulose, preferably consisting of polymethacrylates and ethylcellulose.

Polymethacrylates, ethylcellulose, hydroxyethylcellulose, hydroxypropylcellulose and methylcellulose commonly used in tablet formulations may be used, and reference is made to the extensive literature on suitable polymethacrylates and cellulose derivatives, in particular to Fiedier's “Lexikon der Hilfsstoffe”, 4th ed., ECV Aulendorf (1996), hereinafter referred to as “LdH”, and to “Handbook of Pharmaceutical Excipients”, Wade and Weller, 3rd ed. (2000), hereinafter referred to as “HoPE”, which are incorporated herein by reference.

Preferred retarding agents in oral dosage forms of the invention are, e. g., polymethacrylates having a relative molecular mass of ≧100′000 Da, for example copolymers of acrylic or methacrylic acid esters, e. g. known as Eudragit, for example Eudragit RL 30D (HoPE, page 402), and ethylcellulose, such as Aquacoat®, a 30% by weight aqueous ethylcellulose dispersion available from FMC, or Surelease®, available from Colorcon.

Polymethacrylates can be present in the modified release granules in an amount of from 5 to 25%, preferably from 10 to 20%, e. g. in an amount of about 15%, by weight of the modified release granules.

Ethylcellulose, hydroxyethylcellulose, hydroxypropylcellulose and methylcellulose can be present in the modified release granules in an amount of from 2 to 10%, preferably from 4 to 8%, e. g. in an amount of about 6%, by weight of the modified release granules.

In a further aspect the present invention relates to pharmaceutical oral controlled release compositions comprising licarbazepine, characterized in that in use from 70 to 90%, preferably from 80 to 90%, of said licarbazepine are released within 6 hours, indicated in standard in-vitro dissolution tests at 37° C. in phosphate buffer preferably having a pH of about 6.8 for a 500 mg dosage form.

Clinical studies, for instance, may be effected in a conventional manner. For example, they may be effected over 7 or more days using a 500 mg dose of a compound of the invention. Conveniently at least 6, e. g 10, subjects are enrolled. In such studies modified release characteristics, bioavailability, food effect, safety, tolerability, Cₘₐₓ and/or AUC of the oral dosage forms of the invention can be determined.

The bioavailability of a drug substance depends on its physicochemical properties, such as solubility, and pharmacokinetic properties, e. g. site, rate and extent of absorption. Further, it is known, that food induces changes in the physiology of the gastrointestinal (GI) tract.

These changes can result inter alia in delays in gastric emptying, stimulation of bile flow and changes in pH. Food can also alter lumenal metabolism and physically or chemically interact with a drug substance. It is not surprising, therefore, that food can also effect the bioavailability of a drug substance. The term “food effect” as used herein means, that the bioavailability of a drug substance in a subject in the fed state differs from the bioavailability of this drug substance in a subject in the fasted state. The effects of food are complicated and difficult to predict and will depend, for example, on the nature of the meal, e. g. its nutrient content, fluid volume, caloric content and temperature. It follows, that the presence or absence of a food effect for a given drug substance can only be determined after exhaustive testing.

It is undesirable, if the bioavailability of a drug substance differs depending upon whether a patient is in a fed or fasted state. This will at least be inconvenient to the patient, who will have to time its medication relative to the taking of meals.

It is surprising, therefore, that it was discovered that an oral dosage form of licarbazepine may be administered to a patient without regard to the condition of the patient, i. e. whether the patient is in a fed or fasted state.

Accordingly, the present invention relates in a further aspect to oral dosage forms of the invention having no food effect when administered to a patient.

In a further aspect the present invention relates to a package comprising an oral dosage form of the invention and, e. g. written, instructions for use, said instructions providing that the oral dosage form may be taken equally by patients who have eaten or who are in a fasted condition.

More particularly, the present invention relates to an oral dosage form of the invention packaged in combination with, e. g. written, instructions, which instructions provide that the oral dosage form may be taken equally with or without food.

The presence or absence of a food effect may be quantified by making AUC measurements and/or Cₘₐₓ measurements according to methods well known in the art. Typically, such measurements are made by taking timed biological fluid samples and plotting the serum concentration of the drug substance, e. g. licarbazepine, against time. The values obtained represent a number of values taken from subjects across a patient population and are, therefore, expressed as mean values over the entire patient population. By comparing the mean AUC and/or Cₘₐₓ values, one can determine whether the drug substance, e. g. licarbazepine, exhibits a food effect.

A “fed” subject conveniently may be considered as a subject, that has fasted for at least 10 hours before having received a standard FDA recognised high fat meal. The drug substance, e. g. licarbazepine, may then be administered with water shortly after completion of the meal, e. g. within 5 minutes thereof. Preferably no food should be taken for a period of, e. g., 4 hours after administration of the drug substance, e. g. licarbazepine, although small quantities of water may be permitted after, e.g., 2 hours after administration of the drug substance, e. g. licarbazepine.

A “fasted” subject conveniently may receive the drug substance, e. g. licarbazepine, with water after at least 10 hours of fasting. Thereafter, no food may be taken for a period of, e.g., 4 hours, although small quantities of water may be taken after, e.g., 2 hours after administration of the drug substance, e. g. licarbazepine.

A “standard FDA recognised high fat meal” as referred to herein may comprise any meal, that would be expected to provide maximum perturbation due to the presence of food in the GI tract. Said high fat meal typically may comprise 50% of its caloric value in fat. A representative example may be 2 eggs fried in butter, 2 stripes of bacon, 2 slices of toast with butter, 4 ounces of fried potatoes and 8 ounces of milk.

To study the effect of food on the bioavailability of a drug substance one may use any conventional study design known in the art, for example a randomised, balanced single-dose, two-treatments, two-periods, two-sequences, crossover design. The analysis may be carried out using software from the SAS institute, Cary, N.C., e. g. SAS PROC GLM.

A suitable study design to determine the bioavailability, including the food effect, of an oral dosage form of the invention would be a randomized, open-label, single oral dose, crossover study, wherein one can compare the bioavailability of the oral dosage form of the invention comprising a compound of the invention with the bioavailability of a solution of the same compound of the invention, optionally also including oxcarbazepine film coated tablets, and evaluate the food effect in healthy male subjects being in a fed or fasted state.

In a study wherein the drug substance is, for instance, licarbazepine, the oxcarbazepine film coated tablet (600 mg) and the oral dosage form of the invention comprising, e. g., 500 mg of licarbazepine can be administered together with 240 ml of tap water to the subjects. The licarbazepine clinical service form (500 mg) delivered as powder has to be solubilized in the tap water prior to the drug administration. During the treatment periods that require fasted conditions, the single dose of the study drugs is administered after an overnight fast of at least 10 hours. During the treatment periods that require fed conditions, each subject is requested to eat a standard FDA recognised high fat breakfast within 30 minutes prior to the drug administration. No breakfast is served prior to the drug administration during the treatment periods that require fasted conditions, and the subjects have to continue to fast until 4 hours postdose. The safety and tolerability monitoring includes continuous monitoring of adverse events, physical examinations, blood pressure and pulse rate measurements, ECG recordings and routine laboratory tests (blood chemistry, urinalysis and hematology).

During a first 7 days period, the subjects will be given one of the oral dosage forms of the invention under fasted conditions, and during the second period the subjects will be given the same treatment under fed conditions. The subjects will fast overnight for a minimum of 10 hours on the evening prior to the first dosing of a compound of the invention (period 1). Following dosing at. e. g, breakfast time, pharmacokinetic blood samples may be drawn and used for assays at adequate time intervals, e. g. 0.5, 1, 2, 3, 4, 6, 8, 10, 12, 14, 16, 18, 20, 22, 24, 32 and 48 hours after administration.

The absorption profile of the compound of the invention may be quantified by making AUC measurements on single doses or at the steady state.

Constant plasma levels of the compound of the invention indicate, that the plasma levels of the compound of the invention show low fluctuation indices. The Cₘᵢₙ and Cₘₐₓ values of the compound of the invention may be kept within a small range. To measure the fluctuation between Cₘᵢₙ and Cₘₐₓ, the compound of the invention plasma levels are measured at the steady state, and the fluctuation index is calculated according to (Cₘₐₓ-Cₘᵢₙ)/Cₐᵥ (wherein Cₘₐₓ is the maximum concentration, Cₘᵢₙ is the minimum concentration and Cₐᵥ is the average concentration, observed within a certain time interval, e. g. 24 hours, at the steady state).

The low fluctuation of Cₘᵢₙ and Cₘₐₓ may avoid peak values of the compound of the invention plasma levels, which can be toxic for the patient. A lower fluctuation may provide better tolerablility and safety for the patient treated with a compound of the invention.

Accordingly, in a further aspect the present invention relates to a method of reducing the intra-subject variability of the bioavailability levels of licarbazepine in a patient during oral licarbazepine therapy, said method comprising administering an oral dosage form of the invention comprising licarbazepine as drug substance, which shows no food effect when administered to such patient indiscriminately in the fed or fasted state, e. g. at any hour.

In a further aspect the present invention relates to the use of licarbazepine for the preparation of a medicament for the treatment of patients with affective disorders.

The term “affective disorders” as used herein includes, but is not limited to, uni- and bipolar depression, bipolar disorder, pre-menstrual dysphoric disorder, post-partum depression, post-menopausal depression, neurodegeneration-related depressive symptoms, depression occurring following cessation of psychostimulant intake, psychotic states, e. g. mania, schizophrenia and excessive mood swings where behavioural stabilization is desired.

The utility of the oral dosage forms of the invention for the treatment of affective disorders may be observed in standard animal tests or in standard clinical studies, for example in clinical studies in bipolar disorder patients, with administration of, for example, dosages of licarbazepine in the range of from about 500 to about 3000 mg per day.

The oral dosage forms of the invention may be produced in conventional manner by mixing the components. The resultant mixture may be in powder form, which may be pressed to form a tablet in conventional tabletting machines.

Conveniently oral dosage forms of the invention may be produced by compressing a compound of the invention with, e. g. conventional, tabletting excipients to form a tablet core using conventional tabletting processes and subsequently coating the core. The tablet cores can be produced using conventional granulation methods, for example wet or dry granulation, with subsequent compression and coating. Granulation methods are described, for example, in R. Voigt, “Lehrbuch der Pharmazeutischen Technologie”, Verlag Chemie, 6th edition, pages 156-169.

Granules may be produced in a manner known per se, for example using wet granulation methods known for the production of “built-up” granules or “broken-down” granules.

Methods for the formation of built-up granules may comprise, for example, simultaneously spraying the granulation mass with granulation solution and drying, for example in a drum granulator, in a pan granulator, on a disc granulator or in a fluidised bed, by spray-drying or spray-solidifying, or operate discontinuously, for example in a fluidised bed, in a batch mixer or in a spray-drying drum.

Depending on the method used, the granulation mass may be in the form of a premix or, e. g., may be obtained by mixing a compound of the invention with one or more excipients. The wet granules are preferably dried, for example by tray drying or in a fluidised bed.

The granules thus obtainable may be coated. Suitable coating materials include those conventionally used in coating tablets, granules, or the like. In a preferred embodiment, the coating is water soluble. In another preferred embodiment, the coating is gastric juice resistant, but soluble in intestinal juices.

The oral dosage forms of the invention may contain, in addition to a compound of the invention and at least one retarding agent and at least one disintegrant, conventional excipients depending on the exact nature of the formulation. Suitable categories of excipients include fillers, lubricants, film coating agents, binders, glidants, solubilizers and surface-active substances.

Excipients disclosed in the literature, for instance in LdH or HoPE, the contents of which are incorporated herein by reference, may be used in the pharmaceutical compositions according to the invention. Conveniently the excipients comprise not more than 50%, preferably not more than 40%, more preferably 35%, by weight of the total composition.

An example of suitable fillers is microcrystalline cellulose, which is preferably present in the pharmaceutical compositions according to the invention. Examples include Avicel® (FMC), for example Avicel® PH101, 102, 105, RC581 or RC 591 (LdH, page 216), Emcocel® (Mendell Corp.), Elcema® (Degussa), Filtrak®, Heweten® and Pharmacel®. Preferably the weight ratio of microcrystalline cellulose to the compounds of the invention is from about 1:8 to about 1:14, more preferably from about 1:10 to about 1:12.

Examples of suitable disintegrants include: (i) natural starches, such as maize starch, potato starch, and the like, directly compressible starches, e. g. Sta-rx® 1500, modified starches, e. g. carboxymethyl starches and sodium starch glycolate, available as Primojel®, Explotab® and Explosol®, and starch derivatives, such as amylose; (ii) crosslinked sodium carboxymethylcellulose (croscarmellose sodium), e. g. Ac-di-sol®, Primellose®, Pharmacel® XL, Explocel® and Nymcel® ZSX; (iii) alginic acid and alginates, e. g. sodium alginate; (iv) methacrylic acid-divinylbenzene copolymer salts, e. g. Amberlite® IRP-88; (v) polymers of vinylpyrrolidone, e. g. crosslinked polyvinylpyrrolidones, such as crospovidones, Polyplasdone® XL (LdH, page 1245) and Kollidone® CL; and (vi) magnesium aluminium silicate and bentonite. In a preferred embodiment of the invention, natural starch, e. g. maize starch, and/or croscarmellose sodium are used.

Examples of suitable glidants include: silica, colloidal silica, e. g. Aerosil 200 (LdH, page 117), magnesium trisilicate, powdered cellulose, starch, talc and tribasic calcium phosphate. Colloidal silica, e. g. Aerosil 200, is preferably present in the pharmaceutical compositions according to the invention.

Examples of suitable lubricants include: stearic acid, magnesium stearate, calcium stearate, zinc stearate, talc, polyethylene glycol, sodium benzoate, sodium n-dodecyl sulfate (LdH, page 517), mineral oil and polyoxyethylene monostearate. A combination of lubricants may also be used. Magnesium stearate is preferably present in the pharmaceutical compositions according to the invention.

Examples of suitable surfactants include: alkyl sulfates, such as n-dodecyl sulfates, for example potassium, magnesium or, preferably, sodium n-dodecyl sulfate, e. g. Duponol® C (LdH, page 517), n-tetradecyl sulfates, n-hexadecyl sulfates or n-octadecyl sulfates, non-ionic surfactants of the fatty acid polyhydroxy alcohol ester type, such as sorbitan monolaurate, sorbitan monooleate, sorbitan monostearate, sorbitan monopalmitate, sorbitan tristearate or sorbitan trioleate, polyoxyethylene adducts of fatty acid polyhydroxy alcohol esters, such as polyoxyethylene sorbitan monolaurate, polyoxyethylene sorbitan monooleate, polyoxyethylene sorbitan monostearate, polyoxyethylene sorbitan monopalmitate, polyoxyethylene sorbitan tristearate or polyoxyethylene sorbitan trioleate, polyethylene glycol fatty acid esters, such as polyoxyethyl stearate, polyethylene glycol 400 stearate or polyethylene glycol 2000 stearate, phosphatides, such as lecithin, acacia, tragacanth, polyoxyethylated fats, polyoxyethylated oleotriglycerides, linolizated oleotriglycerides, polyethylene oxide condensation products of fatty alcohols, alkylphenols or fatty acids or 1-methyl-3-(2-hydroxyethyl)-imidazolid-2-one. The term “polyoxyethylated” means, that the substances in question contain polyoxyethylene chains, the degree of polymerization of which generally lies between 2 and 40, in particular between 10 and 20. An alkyl sulfate is preferred.

Oral dosage forms of the invention may be combined with immediate release systems. A combination may be a double-layer tablet comprising an immediate release system and a matrix system with sustained release properties. A double-layer tablet may comprise two doses of the compounds of the invention, e. g. licarbazepine, one part being adapted to provide a sustained release dose and another part adapted to provide an immediate release dose. For tablets comprising licarbazepine, by immediate release is meant release of at least 90% of the dose within 0.5 hours and 100% of the dose within 1.5 hours under the in-vitro licarbazepine test dissolution conditions of the invention.

In one embodiment of the invention, preferably a 500 mg licarbazepine dose is used.

Furthermore, the invention relates to:
A disintegrating tablet having modified release granules comprising licarbazepine and at least one polymer as retarding agents adapted to be administered once a day; in particular such disintegrating tablet wherein the at least one polymer is selected from polymethacrylates, ethylcellulose, hydroxyethylcellulose, hydroxypropylcellulose and methylcellulose. Such disintegrating tablet preferably has no food effect.
A pharmaceutical oral controlled release composition comprising licarbazepine displaying a plateau profile between about 4 and 25 hours after administration.
A method of orally administering licarbazepine for the treatment of affective disorders, said method comprising orally administering to a patient in need of licarbazepine therapy once-a-day an oral dosage form of the invention.

It follows a description, by way of example only, of preferred compositions and processes of the invention.

Abbreviations

Ac acetyl
aqu. aqueous
dansyl 5-(dimethylamino)-1-naphthalenesulfonyl
Et ethyl
HPLC high pressure liquid chromatography
Me methyl
NMR nuclear magnetic resonance
RT or rt room temperature
THF tetrahydrofuran
Ts tosyl

### EXAMPLE 1

### Disintegrating Tablet Comprising Licarbazepine in Modified Release Granules 500 mg of Drug Substance (Coarse Drug Substance; x₅₀ =220 μm) Are Employed Per Tablet

a) Tablet Composition (tablet weight: 790 mg)

Components (in mg)

| | |
|---|---|
| Licarbazepine | 500.00 |
| Eudragit E30D | 100.00 |
| Ethylcellulose, aqueous | 35.00 |
| dispersion | |
| Microcrystalline cellulose | 45.00 |
| Croscarmellose sodium | 40.00 |
| Starch Sta-rx ® | 62.50 |
| Aerosil 200 | 3.75 |
| Magnesium stearate | 3.75 |

b) Preparation

The licarbazepine is spray-granulated in a fluid bed dryer (Aeromatic Fielder MP1) using a dispersion of Eudragit E30D and ethylcellulose. The granules are dried and screened using a Frewitt mill equipped with 1 mm mesh. The microcrystalline cellulose, the croscarmellose sodium, the starch and the Aerosil 200 are also screened and added to the granules. The blend is mixed using a bin blender (Turbula). The magnesium stearate is screened through a hand screen (0.8 mm mesh) and also added. The final blend is mixed using a bin blender (Turbula) and compressed using a Korsch PH250 tabletting press. The tablets are ovaloid, curved, 18 mm long, 7.1 mm wide and without a breaking bar (tablet weight: 790 mg).

### EXAMPLE 2

### In a Manner Analogous to that Described in Example 1, Also Disintegrating 5 Tablets Comprising Licarbazepine in Modified Release Granules can be Prepared, which Comprise 750 mg, 250 mg or 125 mg of Drug Substance Per Tablet

### EXAMPLE 3

### Enantioselective Transfer Hydrogenation of 10-oxo-10,11-dihydro-dibenzo[b,f]azepine-5-carboxylic acid amide to R(−)-10,11-dihydro-10-hydroxy-5H-dibenz[b,f]azepine-5-carboxamide

To a mixture of 10-oxo-10,11-dihydro-dibenzo[b,f]azepine-5-carboxylic acid amide (300 mg, 1.189 mmol) and RuCl[(1R,2R)-p-TsNCH(C₆H₅)CH(C₆H₅)NH₂](η⁶-p-cymene), Aldrich, Switzerland) (8.8 mg, 0.0138 mmol) in CH₂Cl₂ (15 ml) is added dropwise a premixed solution of formic acid and NEt₃ (5:2, 328 mg:289 mg) at 23° C. and stirred for 10 min. The clear solution is heated to reflux for 16 h. The reaction mixture is cooled to RT, diluted with CH₂Cl₂ (20 ml) and neutralised with aqu. NaHCO₃. After washing with brine, the solution is concentrated under reduced pressure. The residue is purified by flash chromatography on silica gel using a 6:1 EtOAc-MeOH mixture as eluent to afford R(−)-10,11-dihydro-10-hydroxy-5H-dibenzo[b,f]azepine-5-carboxamide (enantiomeric purity (ee)>99%, determined by HPLC on Chiracel OD, retention time 9.46 min). [α]_{D}^{rt}=−195.3° (ethanol). ¹H-NMR (400 MHz, CDCl₃): 7.70-7.20 (m, 8H), 5.30 (br s, 1H), 5.10-4.60 (br s, 2H), (m, 1H), 3.20-2.90 (m, 1H), 2.50 (br s, 2H). NMR-data refer to Lit.: Benes, J. et al., J. Med. Chem. 1999, 42, 2582-2587. Molecular weight: 254.291.

### EXAMPLE 4

### Enantioselective Transfer Hydrogenation of 10-oxo-10,11-dihydro-dibenzo[b,f]azepine-5-carboxylic acid amide to S(+)-10,11-dihydro-10-hydroxy-5H-dibenz[b,f]azepine-5-carboxamide

To a mixture of 10-oxo-10,11-dihydro-dibenzo[b,f]azepine-5-carboxylic acid amide (300 mg, 1.189 mmol) and RuCl[(1S,2S)-p-TsNCH(C₆H₅)CH(C₆H₅)NH₂](η⁶-p-cymene) (11mg, 0.0173 mmol) in CH₂Cl₂ (15 ml) is added in two portions a premixed solution of formic acid and NEt₃ (5:2, 656 mg:578 mg) at 23° C. and stirred for 10 min. After that formic acid is added (50 μl) and the clear solution is heated to reflux for 16 h. The reaction mixture is cooled to RT, diluted with CH₂Cl₂ (20 ml) and neutralised with aqu. NaHCO₃. After washing with brine, the solution is concentrated under reduced pressure. The residue is purified by flash chromatography on silica gel using a 6:1 EtOAc-MeOH mixture as eluent to afford S(+)-10,11-dihydro-10-hydroxy-5H-dibenzo[b,f]azepine-5-carboxamide (ee>99% by HPLC on Chiracel OD, retention time 12.00 min). [α]_{D}^{rt}=+196.6° (ethanol). ¹H-NMR (400 MHz, CDCl₃): 7.70-7.20 (m, 8H), 5.30 (br s, 1H), 5.10-4.60 (br s, 2H), 3.75-3.40 (m, 1H), 3.20-2.90 (m, 1H), 2.50 (br s, 2H). NMR-data refer to Lit.: Benes, J. et al., J. Med. Chem. 1999, 42, 2582-2587. Molecular weight: 254.291.

Alternative Process:

To a mixture of 10-oxo-10,11-dihydro-dibenzo[b,f]azepine-5-carboxylic acid amide (300 mg, 1.189 mmol) and RuCl[(1S,2S)-p-dansylNCH(C₆H₅)CH(C₆H₅)NH₂](η⁶-p-cymene) (8.5 mg, 0.012 mmol) in CH₂Cl₂ (15 ml) is added dropwise a premixed solution of formic acid and NEt₃ (5:2, 328 mg:289 mg) at 23° C. and stirred for 10 min. The clear solution is heated to reflux for 16h. The reaction mixture is cooled to RT, diluted with CH₂Cl₂ (20 ml) and neutralised with aqu. NaHCO₃. After washing with brine the solution is concentrated under reduced pressure. The residue is purified by flash chromatography on silica gel using a 6:1 EtOAc-MeOH mixture as eluent to afford S(+)-10,11-dihydro-10-hydroxy-5H-dibenzo[b,f]azepine-5-carboxamide.

### EXAMPLE 5

### RuCl[(1S,2S)-p-dansylNCH(C₆H₅)CH(C₆H₅)NH₂](η⁶-p-cymene)

a) (S,S)-5-dimethylamino-naphthalene-1-sulfonic acid (2-amino-1,2-diphenyl-ethyl)-amide

To a solution of (S,S)-diphenylethylenediamine (250 mg, 1.2 mmol) and triethylamine (0.5 ml) in THF is added dropwise a solution of dansyl chloride (318 mg, 1.2 mmol) in THF (2 ml) at 0° C. After stirring for 16 h at RT the solvent is removed in vacuum and the residue is resolved in methylenchloride (20 ml). The organic solution is washed with NaHCO₃ solution (5 ml), dried over Na₂SO₄, and after filtration the solvent is removed. Flash chromatography affords (S,S)-5-dimethylamino-naphthalene-1-sulfonic acid (2-amino-1,2-diphenyl-ethyl)-amide as yellow oil, which crystallizes by drying in vacuum. Molecular weight: 445.59. ¹H-NMR (400 MHz, CDCl₃): 8.36 (t, J=7.5 Hz, 2H), 8.17 (dd, J=7.2 Hz, 1.2 Hz, 1H (dd, J=8.8 Hz, 1H), 7.34 (dd, J=8.5 Hz, 1H), 7.24-7.16 (m, 4H), 7.11(d, J=7.5 Hz, 1H) 6.99-6.74 (m, 6H), 4.61 (d, J=8.5 Hz, 1H), 4.20 (d, J=8.5 Hz, 1H), 2.80 (s, 6H).

b) RuCl[(1S, 2S)-p-dansylNCH(C6H₅)CH(C₆H₅)NH₂](η⁶-p-cymene)

A solution of (S,S)-5-dimethylamino-naphthalene-1-sulfonic acid (2-amino-1,2-diphenyl-ethyl)-amide (80mg, 0.18 mmol), NEt₃ (36 mg, 0.36 mmol) and [RuCl₂(p-cymene)]₂ (55 mg, 0.09 mmol) in 2-propanol is heated at 80° C. for 1 h. The solvent is removed after that, and the dark red residue is washed with water (2 ml). The solid is dried in vacuum and used without any purification. M: 715.34.

1. A pharmaceutical oral controlled release composition comprising 10,11-dihydro-10-hydroxy-5H-dibenz[b,f]azepine-5-carboxamide.

2. The pharmaceutical composition according to claim 1 being a disintegrating tablet with modified release granules comprising 10,11-dihydro-10-hydroxy-5H-dibenz[b,f]azepine-5-carboxamide.

3. The pharmaceutical composition according to claim 2 wherein the 10,11-dihydro-10-hydroxy-5H-dibenz[b,f]azepine-5-carboxamide is present in the granules in an amount of from 60 to 90% by weight of the modified release granules.

4. A pharmaceutical composition according to claim 2 comprising 10,11-dihydro-10-hydroxy-5H-dibenz[b,f]azepine-5-carboxamide having a median particle size between 150 and 300 μm.

5. A pharmaceutical composition according to claim 2 wherein the modified release granules comprise as retarding agent at least one polymer selected from polymethacrylates, ethylcellulose, hydroxyethylcellulose, hydroxypropylcellulose and methylcellulose.

6. A pharmaceutical composition according to claim 5 comprising polymethacrylates and ethylcellulose wherein the polymethacrylates are present in the granules in an amount of 5 to 25% by weight of the modified release granules.

7. A pharmaceutical composition according to claim 5 comprising polymethacrylates and ethylcellulose wherein the ethylcellulose is present in the granules in an amount of from 2 to 10% by weight of the modified release granules.

8. A pharmaceutical composition according to claim 1 comprising 10,11-dihydro-10-hydroxy-5H-dibenz[b,f]azepine-5-carboxamide in an amount of from 50 to 80% by weight of the total composition.

9. A pharmaceutical composition according to claim 1 comprising microcristalline cellulose.

10. A pharmaceutical composition according to claim 1 comprising at least one natural starch as disintegrant.

11. The pharmaceutical composition according to claim 10 comprising maize starch as disintegrant.

12. A pharmaceutical oral controlled release composition comprising 10,11-dihydro-10-hydroxy-5H-dibenz[b,f]azepine-5-carboxamide, wherein in use 70 to 90% of said 10,11-dihydro-10-hydroxy-5H-dibenz[b,f]azepine-5-carboxamide are released within 6 hours indicated in standard in-vitro dissolution tests at 37° in phosphate buffer having a pH of about 6.8 for a 500 mg dosage form.

13. The pharmaceutical oral controlled release composition comprising 10,11-dihydro-10-hydroxy-5H-dibenz[b,f]azepine-5-carboxamide according to claim 12, wherein in use 80 to 90% of said 10,11-dihydro-10-hydroxy-5H-dibenz[b,f]azepine-5-carboxamide are released within 6 hours indicated in standard in vitro dissolution tests at 37° in phosphate buffer having a pH of about 6.8 for a 500 mg dosage form.

14. A pharmaceutical oral controlled release composition according to claim 1 having no food effect.

15. A disintegrating tablet having modified release granules comprising 10,11-dihydro-10-hydroxy-5H-dibenz[b,f]azepine-5-carboxamide and at least one polymer as retarding agent adapted to be administered once a day.

16. The disintegrating tablet according to claim 15 wherein the at least one polymer is selected from polymethacrylates, ethylcellulose, hydroxyethylcellulose, hydroxypropyl-cellulose and methylcellulose.

17. A disintegrating tablet according to claim 15 having no food effect.

18. A pharmaceutical oral controlled release composition comprising 10,11-dihydro-10-hydroxy-5H-dibenz[b,f]azepine-5-carboxamide displaying a plateau profile between about 4 and 25 hours after administration.

19. Use of 10,11-dihydro-10-hydroxy-5H-dibenz[b,f]azepine-5-carboxamide and excipients as defined in claim 1 for the preparation of a medicament for the treatment of patients with affective disorders.

20. A method of orally administering 10,11-dihydro-10-hydroxy-5H-dibenz[b,f]azepine-5-carboxamide for the treatment of affective disorders, said method comprising orally administering to a patient in need of 10,11-dihydro-10-hydroxy-5H-dibenz[b,f]azepine-5-carboxamide therapy once a day a pharmaceutical composition according to claim 1.
